(19) United States
(12) Patent Application Publication
(10) Pub. No.: US 2026/0217715 A1
(43) Pub. Date: Jul. 30, 2026

(54) AN EFFICIENT PROCESS FOR THE SYNTHESIS OF THEACRINE

(71) Applicant: FERTIS INDIA PVT LTD, Hyderabad, Telangana (IN)

(72) Inventors: Rahul Raju KANUMURU, Hyderabad (IN); Shaji George KOCHUMALAYIL, Hyderabad (IN); Ravikumar SURANENI, Hyderabad (IN); Mohanarao VANA MURALI, Hyderabad (IN)

(73) Assignee: FERTIS INDIA PVT LTD, Hyderabad, Telangana (IN)

(21) Appl. No.: 19/147,645

(22) PCT Filed: Jul. 5, 2023
(86) PCT No.: PCT/IN2023/050654

§ 371 Date: Jul. 11, 2025

(30) Foreign Application Priority Data
Jan. 17, 2023 (IN) 202341003373

(51) Int. Cl. C07D 473/14

(52) U.S. Cl. CPC C07D 473/14

(57) ABSTRACT

The present invention provides an industrial process for the synthesis of Theacrine with high purity and quality.

### CROSS REFERENCE TO RELATED APPLICATIONS

This application is a 35 U.S.C. § 371 National Stage of International Patent Application No. PCT/IN2023/050654, filed Jul. 5, 2023, claiming benefit from Indian Patent Application number 202341003373, filed Jan. 17, 2023, the disclosures of which are incorporated herein in their entirety by reference, and priority is claimed to each of the foregoing.

### FIELD OF THE INVENTION

The present invention relates to an industrial process for the synthesis of Theacrine with high purity and quality.

### BACKGROUND OF THE INVENTION

Theacrine, a tetramethyl uric acid, is a caffeine metabolite present at low levels in various Coffee plants. Chemically, Theacrine is 1,3,7,9-tetramethyl-1H-purine-2,6,8 (3H,7H,9H)-trione having the following structure;

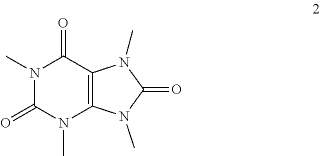 [SMILES: Cn1c(=O)c2c(n(C)c1=O)n(C)c(=O)n2C]

In recent years, studies have found that due to structural similarities to the methylxanthine, caffeine and Theacrine possess similar physiological properties but without undesirable stimulant effects. The available toxicological information on the molecule shows that Theacrine does not cause any genotoxicity and possible health hazards and hence there is continuous interest in Theacrine as an ingredient in functional foods and dietary supplements.

This purine alkaloid is low in content in coffee plants and the extraction process of the same is cumbersome. From the available reports in the literature, it is observed that biotransformation of natural raw materials such as caffeine into Theacrine compounds is expensive, the efficiency is low, and it is difficult to commercialize due to poor atom economy.

The present inventors therefore felt that the unfilled gap in the production of Theacrine (2) can be fulfilled by providing an industrial method for the synthesis whereby Theacrine can be achieved in sufficient quantities for industrial applications without restriction of raw materials.

### SUMMARY OF THE INVENTION

In lieu of the above objective, the present invention provides a process for the synthesis of Theacrine in an efficient manner which process comprises; reacting compound(s) of formula I with a methylating agent in presence of a base to obtain Theacrine (2). The synthetic process is shown in below scheme 1.

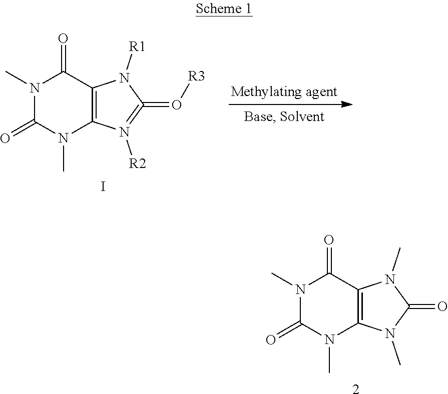

Wherein, R1 is hydrogen or methyl; R2 is hydrogen or absent and; R3 is methyl, dashed bond can be single or double bond,
with the proviso;
when the dashed bond is a double bond to oxygen,
then R1 and R2 is hydrogen and R3 is absent
with the proviso;
when the dashed bond is a single bond to oxygen,
then, R1 and R3 are methyl and R2 is absent.

Accordingly, in one of the embodiments, the compound of formula I is selected from the compound of formula 1 and compound of formula 3, as shown below.

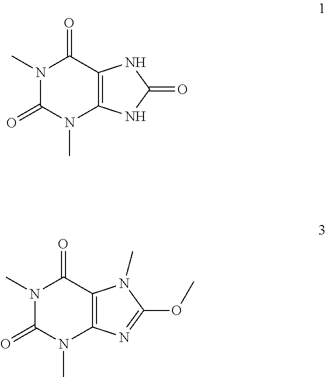 [SMILES: Cn1c(=O)c2[nH]c(=O)[nH]c2n(C)c1=O ; COc1nc2c(c(=O)n(C)c(=O)n2C)n1C]

### DETAILED DESCRIPTION OF THE INVENTION

The present invention will now be explained in detail with reference to its various preferred as well as optional embodiments, which, however, should not be construed to limit the scope of the invention.

In an embodiment, the present invention aims to provide a process for the preparation of Theacrine (2) in an efficient and industrially scalable manner.

In pursuit of the above objective, the present inventors have explored the synthesis of Theacrine by reacting 1,3-dimethyl-1H-purine-2,6,8 (3H,7H,9H)-trione, compound of formula 1 with variety of methylating agent(s) in presence of variety of bases and solvents to afford Theacrine (2).

Accordingly, in an embodiment, the methylation reaction was explored using methylating agents selected from the group consisting of dimethyl sulphate, DMF-DMA, methyl iodide, Trimethyl phosphate and the like.

In another embodiment, the methylation reaction was conducted in presence of various organic and inorganic bases. More preferably, the methylation reaction was conducted with an inorganic base selected from the group consisting of sodium hydroxide, sodium hydride, potassium carbonate and the like.

In yet another embodiment, the methylation reaction also explored in presence of various solvents selected from the group consisting of alcoholic solvents such as methanol, water, acetone, DMF, DMA, NMP etc.

In yet another embodiment, the methylation reaction is carried at various temperature conditions ranging from 50° C. to 170° C.

The results of various embodiments of methylation reaction of the compound of formula 1 is disclosed in table 1. The methylation reaction is shown in scheme 2.

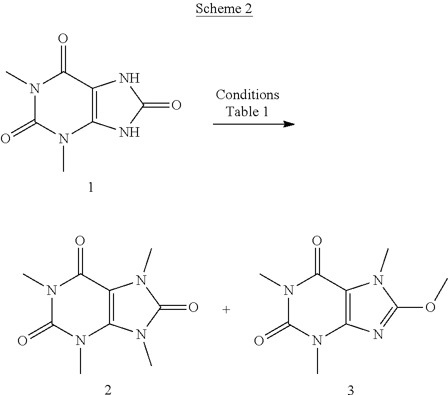

TABLE 1

| | | Time | | |
|---|---|---|---|---|
| S. No | Conditions | (h) | 2 (%) | 3 (%) |
| 1 | DMS, NaOH, MeOH, water, 50° C. | 6 | 80 | 14 |
| 2 | DMS, K₂CO₃, Acetone, 60° C. | 24 | 88 | 12 |
| 3 | DMF-DMA, 150° C., 24 h | 24 | 70 | 20 |
| 4 | MeI, NaH, DMF | 6 | 55 | 25 |
| 5 | Trimethyl phosphate, K₂CO₃, 150° C. | 12 | 78 | 18 |
| 6 | Trimethyl phosphate, DMF, K₂CO₃, 150° C. | 5 | 99 | 0.1 |

As is evident from the scheme 2 and table 1 (Serial no 1-5), methylation of trione intermediate (1) using various methylating agents and reaction conditions to afford Theacrine (2), resulted in the formation of compound (3) as impurity in varying proportions ranging from 10 to 25%. Presence of compound 3 in the product results in purification difficulties, loss of atom economy and thus additional cost.

Therefore, in one of the preferred embodiments, the invention provides a process for preparation of Theacrine, which process comprises; Methylating compound (1) with trimethyl phosphate in presence of a base to obtain Theacrine (2). In an embodiment, the base is selected from the group consisting of sodium hydroxide, sodium hydride and potassium carbonate.

In another embodiment, the reaction is conducted in presence of a solvent selected from the group consisting of methanol, acetone, dimethylformamide, dimethyl sulfoxide, dimethyl acetamide, N-Methyl pyrrolidine, acetonitrile, ethyl acetate etc.

In one of the preferred embodiments, the solvent is dimethylformamide.

In an embodiment, the methylation reaction of compound 1 can be conducted at a temperature range of 50 to 170° C. More preferably, the methylation reaction is carried out at a temperature of 150° C. to achieve Theacrine in good yields and in high purity.

Accordingly, in an embodiment, the present inventors have developed a reaction condition using trimethylphospate as methylating agent in the presence of potassium carbonate base and DMF as solvent at temperature around 150° C., which exclusively results in the formation of Theacrine (2) in high yields and quality, as shown in below scheme 3.

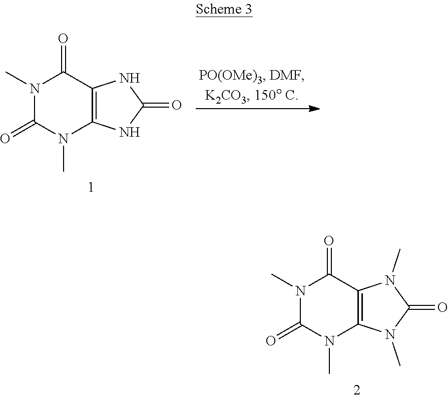

Further, the present inventors have surprisingly found that the mixtures of Theacrine (2) and compound (3) in different proportions obtained (as shown in table 1 under Sr. No 1 to 5) upon subjecting to methylation conditions using trimethylphospate as methylating agent in the presence of potassium carbonate base and DMF as solvent under the temperature conditions of 150° C. resulted in exclusive formation of Theacrine (2) in higher yields, which becomes additional embodiment of the present invention. The process of this embodiment is shown in below scheme 4.

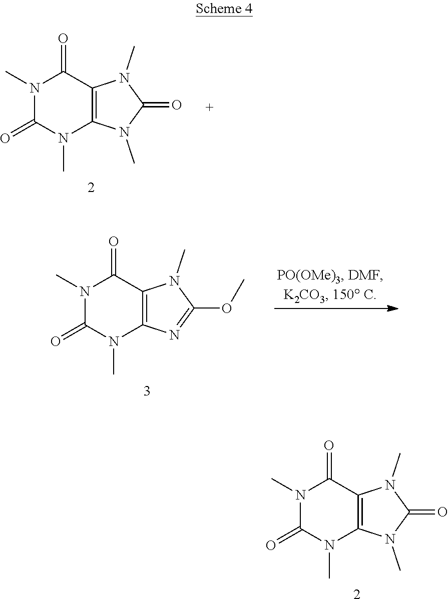

In yet another embodiment, when a mixture of compounds 2 and 3 in solvents such as DMF, DMA, NMP or DMF-DMA or in a mixture of DMF PO(OMe)₃ heated around 150° C.; it was observed that the compound 3 exclusively rearranged to Theacrine 2 in high yields and purity. The rate of reaction further increased in the presence of potassium carbonate.

Encouraged with the above results, the compound 3 was N-methylated in presence of trimethyl phosphate as methylating agent under the conditions reported in Sr. No. 6 in the table 1, exclusively resulted in the formation of Theacrine (2) in higher yields and relatively in shorter reaction times, which becomes additional embodiment of the present invention. The process is depicted in below scheme 5.

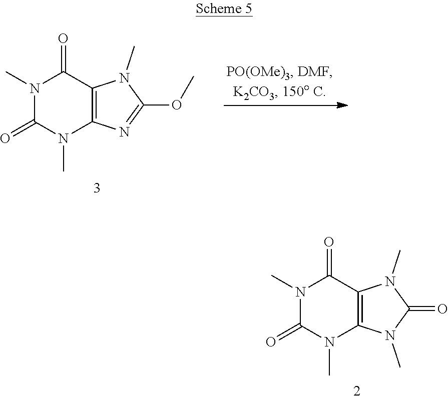

Thus, the present invention provides a process for the preparation of Theacrine (2) in an efficient manner which process comprises one or more of the following;
a) Reacting compound(s) of formula I with a methylating agent in presence of a base to obtain Theacrine (2); or
b) heating a mixture of compounds of formula 1 and 3 in solvents such as DMF, DMA, NMP etc. in the presence of a base preferably potassium carbonate around 150° C. to give Theacrine (2) or
c) heating compounds of formula 3 in solvents such as DMF, DMA, NMP etc. around 150° C. to give Theacrine (2); or
d) optionally purifying the Theacrine from methanol.

The synthetic process is shown in below scheme 1.

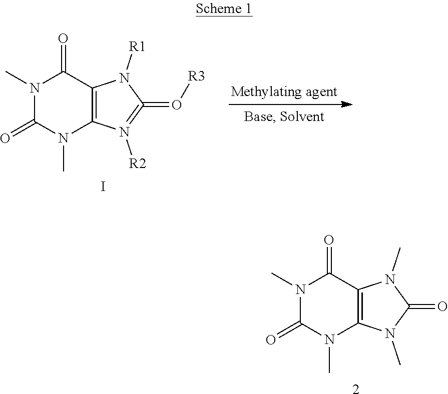

Wherein, R1 is hydrogen or methyl; R2 is hydrogen or absent and; R3 is methyl, dashed bond can be single or double bond,
with the proviso;
when the dashed bond is a double bond to oxygen,
then R1 and R2 is hydrogen and R3 is absent
with the proviso;
when the dashed bond is a single bond to oxygen,
then, R1 and R3 are methyl and R2 is absent.

Accordingly, the compound of formula I is selected from the compound of formula 1 and compound of formula 3, as shown below.

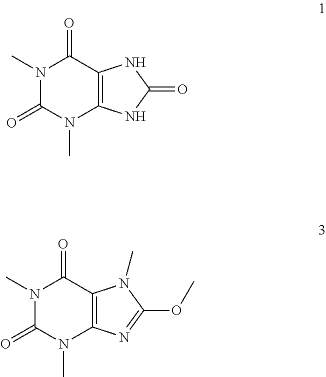 [SMILES: Cn1c(=O)c2[nH]c(=O)[nH]c2n(C)c1=O ; COc1nc2c(c(=O)n(C)c(=O)n2C)n1C]

In an embodiment, the methylating agents are selected from the group consisting of dimethyl sulphate, DMF-DMA, methyl iodide, Trimethyl phosphate and the like. In another embodiment, the methylation reaction is conducted with an inorganic base selected from the group consisting of sodium hydroxide, sodium hydride, potassium carbonate and the like.

In a further embodiment, the solvents that are suitable for the methylation reaction is selected from the group consisting of methanol, water, acetone, DMF, DMA, NMP etc.

The methylation reaction is carried at various temperature conditions ranging from 50° C. to 170° C.

In additional embodiments, the invention provides a process for preparation of Theacrine 2 which process comprises reacting compound(s) of formula 3 in a solvent optionally in presence of potassium carbonate base at 150° C. to obtain Theacrine (2), wherein, the solvents are selected from the group consisting of DMF, DMA and NMP. This reaction is a thermal rearrangement of compound 3 to compound 2, which is facilitated by higher temperature and in the presence of potassium carbonate. Although, this reaction can be performed in the absence of potassium carbonate; the time taken for reaction completion longer.

In further embodiments, the invention provides a process for preparation of Theacrine 2 which process comprises reacting mixtures of compound(s) of formula 2 and formula 3 with Trimethyl phosphate as methylating agent in presence of a solvent and optionally in presence of potassium carbonate base in a solvent at 150° C. to obtain Theacrine (2), wherein, the solvents are selected from the group consisting of DMF, DMA and NMP.

In another embodiment, the Theacrine thus obtained is purified by dissolving in methanol under reflux conditions and cooled to 40° C. The solid thus obtained is washed with hot methanol (40-45° C.) to obtain Theacrine with good purity of more than 99% by HPLC.

Thus, various embodiments of the present invention avoid use of toxic chemicals and solvents and further results in the formation of the end product 2 in good atom economy with yields generally more than 95% and high purity of 99% by HPLC.

The following example, which includes preferred embodiments, will serve to illustrate the practice of this invention, it being understood that the particulars shown are by way of example and for purpose of illustrative discussion of preferred embodiments of the invention.

### EXAMPLES

### Example 1: Preparation of 1,3,7,9-tetramethyl-1H-purine-2,6,8 (3H,7H,9H)-trione, (Theacrine, 2)

To a stirred solution of 1,3-dimethyl-1H-purine-2,6,8 (3H,7H,9H)-trione 1 (25.0 g, 12.8 mmol), in DMF (150 mL) was added potassium carbonate (53.3 g, 38.6 mmol) and trimethyl phosphate (72.2 g, 51.2 mmol). The mixture was stirred at 150° C. After 5 h, DMF removed under vacuum, the residue cooled to room temperature, treated with water and extracted with a mixture of 10% MeOH and DCM (3×200 mL), organic phases combined, washed with brine, dried over Na₂SO₄, filtered and concentrated under vacuum. The crude was dissolved in MeOH (5 vol) and heated at reflux for 2 h, then cooled to 40° C. The solid formed was filtered and washed with hot (40-45° C.) MeOH (1 vol), to afford pure Theacrine (2) as white solid. Yield: 27.5 g (96.7%). ¹HNMR spectrum (400 MHz, DMSO-d6, δ, ppm, J/Hz): 3.19 (3H, s), 3.39 (3H, s), 3.52 (3H, s), 3.63 (3H, s), 3.76 (6H, s). MS (m/z): 225.22 (M+H)⁺. Purity (HPLC): 99.8%

### Example 2: Preparation of 1,3,7,9-tetramethyl-1H-purine-2,6,8 (3H,7H,9H)-trione (Theacrine, 2)

To a mixture of 1,3,7,9-tetramethyl-1H-purine-2,6,8 (3H,7H,9H)-trione (Theacrine, 2) and O-Methoxy compound 3 (5.0 g, 25.7 mmol; 3:1 ratio), in DMF, was added trimethyl phosphate (25 mL) and potassium carbonate (10.7 g, 77.3 mmol). The mixture was stirred at 150° C. for 5 h. After 5 h, it was cooled to room temperature, treated with water and extracted with 10% MeOH/DCM (3×50 mL), the combined organic phase washed with brine, dried over Na₂SO₄, filtered and concentrated under vacuum. The residue dissolved in MeOH (5 vol), heated at reflux for 2 h and then cooled to 40° C. The solid formed was filtered and washed with hot (40-45° C.) methanol (1 vol), to afford pure Theacrine (2) as white solid. Yield: 4.8 g (96.0%). Purity (HPLC): 99.5%

### Example 3: Preparation of 1,3,7,9-tetramethyl-1H-purine-2,6,8 (3H,7H,9H)-trione (Theacrine, 2)

To a stirred solution of O-Methoxy compound 3 (1.0 g, 4.46 mmol), in DMF (5 mL) was added potassium carbonate (1.85 g, 13.38 mmol) the mixture stirred at 150° C. for 5 h. After 5 h, DMF removed under vacuum, the mixture cooled to room temperature, treated with water and extracted with 10% MeOH/DCM (3×20 mL). The organic phases combined, combined phase washed with brine, dried over Na₂SO₄, filtered and concentrated under vacuum. The residue dissolved in MeOH (5 vol) and heated at reflux for 2 h and cooled to 40° C. The solid thus formed was filtered and washed with hot (40-45° C.) MeOH (1 vol), to afford pure Theacrine (2) as a white solid. Yield: 0.95 g (95.0%).

### Example 4: Preparation of 1,3,7,9-tetramethyl-1H-purine-2,6,8 (3H,7H,9H)-trione (Theacrine, 2)

To a stirred solution of compound 3 (1.0 g, 4.46 mmol), in DMA (5 mL) was added potassium carbonate (1.85 g, 13.38 mmol). The reaction mixture was stirred at 150° C. for 8 h. After 8 h, DMA was removed under vacuum, the mixture cooled to room temperature, added water and extracted with 10% MeOH/DCM (3×20 mL). Organic phases combined, combined phase washed with brine, dried over Na₂SO₄ and concentrated under vacuum. The residue dissolved in MeOH (5 vol) and refluxed for 2 h and cooled to 40° C. The solid formed was filtered and washed with hot (40-45° C.) MeOH (1 vol), to afford pure Theacrine (2) as white solid. Yield: 0.96 g (96.0%).

### Example 5: Preparation of 1,3,7,9-tetramethyl-1H-purine-2,6,8 (3H,7H,9H)-trione (Theacrine, 2)

To a stirred solution of compound 3 (1.0 g, 4.46 mmol) in NMP (5 mL) was added potassium carbonate (1.85 g, 13.38 mmol) the mixture stirred at 150° C. for 8 h. After 8 h, NMP removed under vacuum, and the mixture cooled to room temperature. It was treated with water, extracted with 10% MeOH/DCM (3×20 mL), the organic phases combined, combined phase washed with brine, dried over Na₂SO₄, filtered and concentrated under vacuum. The crude was dissolved in MeOH (5 vol) and heated at reflux for 2 h, then cooled at 40° C. The solid formed was filtered and washed with hot (40-45° C.) MeOH (1 vol), to afford pure Theacrine (2) as white solid. Yield: 0.93 g (93.0%).

### Example 6: Preparation of 1,3,7,9-tetramethyl-1H-purine-2,6,8 (3H,7H,9H)-trione (Theacrine, 2)

To a stirred solution of compound 3 (1.0 g, 4.46 mmol) in DMF (5 mL) was stirred at 150° C. for 16 h. After 16 h DMF removed under vacuum, the residue cooled to room temperature. It was treated with water, extracted with 10% MeOH/DCM (3×20 mL) and the organic phases combined and the combined phase washed with brine, dried over Na₂SO₄, filtered and concentrated under vacuum. The residue dissolved in MeOH (5 vol), heated to reflux for 2 h, and then cooled to 40° C. The solid formed was filtered and washed with hot (40-45° C.) MeOH (1 vol), to afford pure Theacrine (2) as white solid. Yield: 0.97 g (97.0%).

The process of the present invention provides suitable reaction conditions, for the synthesis of Theacrine with higher conversion rates and higher yields. The synthesis is convenient, post-processing/work up procedure is simpler, and the method is suitable for large-scale production. Also, the method can be performed with low-cost equipment without the requirement for high pressure conditions; with adoption of simple recrystallization for preparing Theacrine with high purity in an economically feasible way. Thus, the present invention provides relatively high yield of Theacrine in pure form and the process is relatively inexpensive.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A process for the preparation of Theacrine (2) in an efficient manner which process comprises
a) reacting compound(s) of formula I with a methylating agent in presence of base and solvent to obtain Theacrine (2); and optionally purifying the Theacrine from methanol, 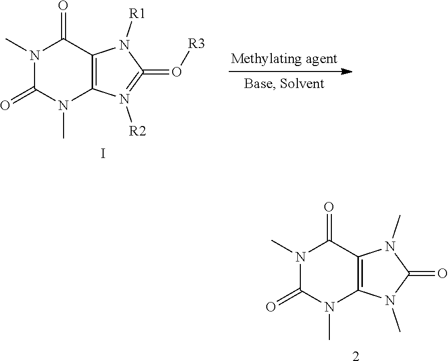
wherein, R1 is hydrogen or methyl; R2 is hydrogen or absent and; R3 is methyl, dashed bond can be single or double bond,
with the proviso;
when the dashed bond is a double bond to oxygen,
then R1 and R2 is hydrogen and R3 is absent
with the proviso;
when the dashed bond is a single bond to oxygen,
then, R1 and R3 are methyl and R2 is absent.

2. The process as claimed in claim 1, wherein, the compound of formula I is selected from the compound of formula 1 and compound of formula 3, as shown below.

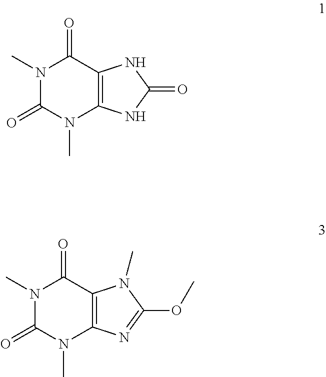 [SMILES: Cn1c(=O)c2[nH]c(=O)[nH]c2n(C)c1=O ; COc1nc2c(c(=O)n(C)c(=O)n2C)n1C]

3. The process as claimed in claim 1, wherein, the methylating agent is selected from the group consisting of dimethyl sulphate, DMF-DMA, DMA, NMP, methyl iodide, Trimethyl phosphate and the like.

4. The process as claimed in claim 1, wherein, the base is selected from the group consisting of sodium hydroxide, sodium hydride, potassium carbonate and the like.

5. The process as claimed in claim 1, wherein, the solvent is selected from the group consisting of methanol, water, acetone, DMF, DMA, NMP etc.

6. The process as claimed in claim 1, wherein, the methylation reaction is carried at temperature conditions ranging from 50° C. to 170° C.

7. The process as claimed in claim 1, wherein, the process comprises reacting compound(s) of formula 1 with Trimethyl phosphate as methylating agent in presence of potassium carbonate base in DMF solvent at 150° C. to obtain Theacrine (2).

8. The process as claimed in claim 1, wherein, the process comprises reacting mixtures of compound(s) of formula 1 and formula 3 with Trimethyl phosphate as methylating agent in presence of potassium carbonate base in DMF solvent at 150° C. to obtain Theacrine (2).

9. The process as claimed in claim 1, wherein, the process comprises reacting compound(s) of formula 3 in a solvent optionally in presence of potassium carbonate base at 150° C. to obtain Theacrine (2).

10. The process as claimed in claim 9, wherein, the solvent is selected from the group consisting of DMF, DMA and NMP.

11. The process for preparation of Theacrine 2 as claimed in claim 9, wherein the process comprises heating compound formula 3 in DMF solvent optionally with potassium carbonate at 150° C. to obtain Theacrine (2).

12. The process for preparation of Theacrine 2 as claimed in claim 9, wherein the process comprises heating compound formula 3 in DMA solvent optionally with potassium carbonate at 150° C. to obtain Theacrine (2).

13. The process for preparation of Theacrine 2 as claimed in claim 9, wherein the process comprises heating compound formula 3 in NMP solvent optionally with potassium carbonate at 150° C. to obtain Theacrine (2).

14. A process for preparation of Theacrine 2 which process comprises reacting mixtures of compound(s) of formula 2 and formula 3 with Trimethyl phosphate in presence of a solvent and optionally in presence of potassium carbonate base at 150° C. to obtain Theacrine (2).

15. The process for preparation of Theacrine 2, as claimed in claim 14, wherein, the solvent is selected from the group consisting of DMF, DMA and NMP.

16. The process for preparation of Theacrine 2, as claimed in claim 14, wherein, the process comprises heating a mixture of compound(s) of formula 2 and formula 3 in DMF solvent optionally in presence of potassium carbonate at 150° C. to obtain Theacrine (2).

17. The process for preparation of Theacrine 2 as claimed in claim 14, wherein the process comprises heating a mixture of compound(s) of formula 2 and formula 3 in DMA solvent optionally in presence of potassium carbonate at 150° C. to obtain Theacrine (2).

18. The process for preparation of Theacrine 2, as claimed in claim 14, wherein, the process comprises heating a mixture of compound(s) of formula 2 and formula 3 in NMP solvent optionally in presence of potassium carbonate at 150° C. to obtain Theacrine (2).

19. The process of claim 1, comprising purifying crude Theacrine 2 by a) dissolving the crude Theacrine 2 in methanol under reflux; b) cooling to 40° C. followed by washing the solid with hot methanol (40-45° C.) to afford pure Theacrine (2).
